(19) United States
(12) Patent Application Publication
(10) Pub. No.: US 2026/0007348 A1
(43) Pub. Date: Jan. 8, 2026

(54) METHOD AND SYSTEM FOR REAL TIME ELECTROCARDIOGRAM WAVEFORM PROCESSING WITH MODE ADAPTIVE COEFFICIENT FILTERS

(71) Applicant: DRÄGERWERK AG & CO. KGAA, Lübeck (DE)

(72) Inventors: Nicholas Zakas, Londonderry, NH (US); Ruwen Zhang, Andover, MA (US)

(21) Appl. No.: 18/992,515

(22) PCT Filed: Jul. 10, 2023
(86) PCT No.: PCT/IB2023/057073

§ 371 Date: Jan. 8, 2025

Related U.S. Application Data
(60) Provisional application No. 63/388,296, filed on Jul. 12, 2022.

(51) Int. Cl. A61B 5/308; A61B 5/00; A61B 5/339

(52) U.S. Cl. CPC A61B 5/308; A61B 5/339; A61B 5/746

(57) ABSTRACT

A mode adaptive coefficient filter system for a physiological patent monitoring (“PPM”) method and apparatus provides an electrocardiogram (“ECG”) display function with an improved resolution of artifact issues when large baseline offsets occur to input signals. In one aspect of the disclosure the method and system provide an ECG monitoring and display function using the mode adaptive coefficient filter system for an active filter process provides for an improved ECG artifact resolution minimizing discontinuous wave forms when applied to an input signal that achieves improved signal quality and reduced latency in signal rendering. The method may be utilized for both infinite impulse response (“IIR”) and finite impulse response (“FIR”) systems with similar filter structures.

### CROSS-REFERENCE TO RELATED APPLICATIONS

The priority and earlier effective filing date of U.S. Application Ser. No. 63/388,296, filed Jul. 12, 2022, is hereby claimed for all purposes, including the right of priority. This related application is also hereby incorporated by reference for all purposes as if expressly set forth verbatim herein.

### TECHNICAL FIELD

The present disclosure pertains to a mode adaptive coefficient filter system and, more specifically to mode adaptive coefficient filters used during an electrocardiogram (“ECG”) procedure.

### DESCRIPTION OF THE RELATED ART

This section of this document introduces information about and/or from the art that may provide context for or be related to the subject matter described herein and/or claimed below. It provides background information to facilitate a better understanding of the various aspects of the that which is claimed below. This is a discussion of “related” art. That such art is related in no way implies that it is also “prior” art. The related art may or may not be prior art. The discussion in this section of this document is to be read in this light, and not as admissions of prior art.

Physiological patent monitoring (“PPM”) products may employ, for example, electrocardiogram (“ECG”) function graphs of voltage acquired from a person's body over time. The voltages represent electrical activity of the heart. To acquire the voltages, electrodes are placed at selected points on the person's body. It is desirable to establish a strong physical contact and electromagnetic coupling between each of the electrodes and the person's body. The strong physical contact and electromagnetic coupling are desirable because they promote good data acquisition that improves the accuracy of the ECG.

### BRIEF DESCRIPTION OF THE DRAWINGS

Illustrative embodiments of the subject matter claimed below will now be disclosed. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

FIG. 1 conceptually depicts an ECG procedure in accordance with the present disclosure.

FIG. 2 conceptually illustrates one particular implementation of the processor-based control unit of FIG. 1.

FIG. 3A-FIG. 3B conceptually illustrate a case where noticeable artifacts were observed on a system display without the mode adaptive coefficient filter design as disclosed herein.

FIG. 4 illustrates the resolution of ECG signal artifacts after mode adaptive coefficient filter improvements are provided by the present disclosure.

FIGS. 5A and 5B conceptually depict selected portions of a mode adaptive coefficient filter system in accordance with the present disclosure.

While the invention is susceptible to various modifications and alternative forms, the drawings illustrate specific examples herein described in detail by way of example. It should be understood, however, that the description herein of specific examples is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

Unfortunately, in ECG procedures, discontinuities and artifacts occur particularly at high or variable ECG voltages, and there is latency in signal rendering such as oscillation or ripples. Currently, attempting to rapidly address such signal errors and difficulties requires either physical signal switching between specific filter modes, all of which fail to correct for discontinuities or artifacts of the actual ECG display baselines, or providing all designated filter modes at the same time which includes the discontinuities or artifacts with no compensation rendering baseline variations in a display without fidelity restoration.

In ECG signal processing, a finite impulse response (“FIR”) filter is a filter whose impulse response is of finite duration, because it settles to zero in a finite time. This is in contrast to infinite impulse response (“IIR”) filters, which may have internal feedback and may continue to respond indefinitely.

A number of detriments in conventional physiological patient monitoring (“PPM”) products employing coefficient filter systems occur when switching between infinite impulse response (“IIR”) filters and finite impulse response (“FIR”) filters and they are not adaptive. These detriments include noticeable oscillation artifacts, dis-continuity in baselines, latency in real time signal rendering, and error in waveform displays.

As alluded to above, mode adaptive coefficient filters are designed and implemented in PPM patient ECG monitoring products and the present invention provides mode adaptive coefficient filters designed based on clinical needs to achieve a variety of physiological signal qualities that address at least one of the concerns noted and achieve ECG artifact resolution. The present mode adaptive coefficient filter system achieves ECG mode artifact resolution by applying selected mode filter parameter controls.

In one embodiment, an electrocardiogram (“ECG”) monitor, comprises a mode adaptive coefficient filter system, a display, and a processor-based control unit. The mode adaptive coefficient filter system is for real time electrocardiogram (“ECG”) waveform processing of ECG waveforms received from a plurality of ECG electrodes and includes at least one active n-order impulse filter. The n-order impulse filter is an infinite impulse response (“IIR”) filter or a finite impulse response (“FIR”) filter. The processor-based control unit includes the mode adaptive coefficient filter system. The processor-based control unit programmed to: monitor a plurality of ECG electrodes when the ECG electrodes are ostensibly electrically connected; receive a physiological signal acquired from one of the ECG electrodes as an electrical input signal including at least one artifact relative to a waveform display baseline; apply the mode adaptive coefficient filter system based on a plurality of preconfigured filter parameter settings; and render an input signal waveform in the display that removes the at least one artifact relative to the waveform display baseline.

In other embodiments, a method for real time electrocardiogram (“ECG”) waveform processing, comprising: monitoring a plurality of electrocardiogram (“ECG”) electrodes in a physiological patent monitoring (“PPM”) product ostensibly electrically connected to a human body and including an active n-order impulse filter, the n-order impulse filter being one of an infinite impulse response (“IIR”) filter and a finite impulse response (“FIR”) filter; receiving a physiological signal acquired from one of the ECG electrodes as an electrical input signal including at least one artifact relative to a waveform display baseline; applying a predetermined mode adaptive coefficient filter based on a plurality of preconfigured filter parameter settings; rendering an input signal waveform in a display; and removing the at least one artifact relative to the waveform display baseline.

In still other embodiments, a non-transitory computer readable medium encoded with instructions that, when executed by a processor, perform any of the methods for real time electrocardiogram (“ECG”) waveform processing disclosed and/or claimed herein.

In yet other embodiments, a mode adaptive coefficient filter system for real time electrocardiogram (“ECG”) waveform processing comprises a plurality of electrocardiogram (“ECG”) electrodes and an ECG monitor. The ECG electrodes may be in a physiological patient monitoring (“PPM”) product and ostensibly electrically connected to a human body. The ECG monitor electrically connected to the ECG electrodes and includes at least one active n-order impulse filter a display, and a processor-based control unit. The at least one active n-order impulse filter may be an infinite impulse response (“IIR”) filter or a finite impulse response (“FIR”) filter. The processor-based control unit includes the at least one filter, a processor, and a memory. In the memory resides a plurality of instructions with which the processor-based control unit is programmed and that, when executed by the processor, cause the processor to apply the mode adaptive coefficient filter system based on a plurality of preconfigured filter parameter settings.

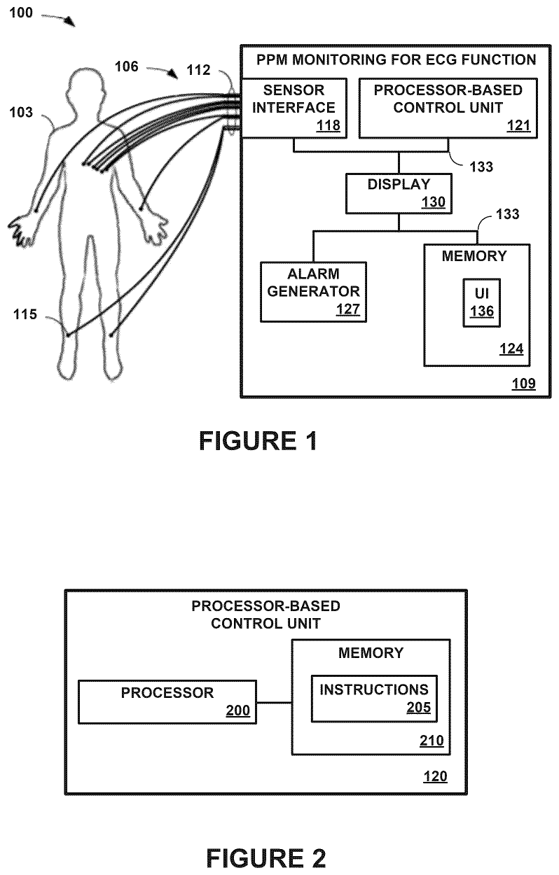

Turning now to the drawings, FIG. 1 is an exemplary illustration of a PPM patient monitoring product in accordance with the present disclosure with a mode adaptive coefficient filter system. In FIG. 1, a patient 103 is undergoing the ECG procedure 100 being administered using the ECG system 106. The ECG system 106 comprises an ECG monitor 109, a plurality of electrical leads 112, and a plurality of ECG electrodes 115 (only two are indicated). There need not be a 1:1 correspondence between the ECG electrodes 115 and the electrical leads 112 as is shown in FIG. 1. The ECG electrodes 115 are ostensibly attached to the patient 103 and connected to the ECG monitor 109 via the electrical leads 112.

As used herein, the term “ostensibly attached” means that the ECG electrodes 115 are intended to physically contact and electromagnetically couple to the body of the patient 103 sufficiently to acquire suitable ECG data during the ECG procedure 100. However, it is possible that one or more of the ECG electrodes 115 may become detached from the body of the patient 103 to a problematical degree. The present disclosure includes, but is not limited to, a technique by which the ECG monitor 109 having a mode adaptive coefficient filter system following the present disclosure may also determine whether ECG electrodes 115 are attached or may be detached. The term “ostensibly attached” describes this situation.

The ECG monitor 109 includes, in this particular example, at least a sensor interface 118, a processor-based control unit 120, a memory 124, and an alarm generator 127. In some embodiments, the memory 124 and alarm generator 127 may be incorporated into the processor-based control unit 120. Although the technique for the mode adaptive coefficient filter system disclosed herein further provides that the ECG monitor 109 also includes a display 130. The display 130 may be used to present a user interface (“UI”, not separately shown) through which a user may interact with the ECG monitor 109. The various components of the ECG monitor 109 may communicate with one another over a bus system 133.

The display 130 may be any suitable type of display known to the art. The display may be, for example and without limitation, a Light Emitting Diode (“LED”) display, a Liquid Crystal Display (“LCD”), an organic light-emitting diode (OLED or organic LED), an electroluminescent display, or even a cathode-ray tube display. The display 130 may be a touch screen with supporting processing in some embodiments and may not be in other embodiments. Embodiments in which the display 130 is a touch screen may be used to present a Graphical User Interface (“GUI”). In embodiments in which the display 130 is not a touch screen, the UI may include mechanical means, such as buttons, keys, and/or switches, to interface with a user. Regardless of whether the display 130 includes a touch screen, various embodiments may also employ a variety of peripheral input devices (not shown) as a part of the UI. Examples of such peripheral input devices include, without limitation, pointing devices such a mouse, trackpad, or track ball and keyboards.

As with the display 130 and the UI, the system disclosed herein also admits wide variation in the implementation of the alarm generator 127. The alarm generator 127 may be, for example, an audio speaker to broadcast an audio alarm. In other examples, the alarm generator 127 may include a light, such as an LED, to provide a visual alarm upon a detection of a noticeable artifact. Note that, in some embodiments, a visual alarm may be presented on the display 130 through the UI such that the alarm generator 127 may be omitted.

The sensor interface 118 receives data from the ECG electrodes 115 over the electrical leads 112 and then conditions that data for use and handling by the rest of the ECG monitor 109. The sensor interface 118 may include, for example, one or more electrical connectors (not separately shown) the receive(s) and/or mates with one or more electrical connectors (also not separately shown) that may comprise a part of the electrical leads 112. The sensor interface 118 may also include electrical circuitry and electronic components for conditioning the received data. As those in the art having the benefit of this disclosure will appreciate, the precise makeup of the sensor interface 118 in any given embodiment will be implementation specific. Factors for consideration may include, without limitation, the quantity, quality, form, and format of the received data.

Those in the art having the benefit of this disclosure will also appreciate that the ECG monitor 109 will typically include other components not separately shown. For example, the ECG monitor 109 may include a battery, a connection to a power supply such as an electrical grid, or both. Similarly, the ECG monitor 109 may also include mechanical buttons or switches for a user to interact with the ECG monitor 109 during use as mentioned above. However, these and other features of the ECG monitor 109 not germane to the practice of the technique disclosed herein have been omitted for the sake of clarity and to promote an understanding of that which is claimed below.

FIG. 2 conceptually illustrates one particular implementation of the processor-based control unit 120 operating with the mode adaptive coefficient filter system. In the illustrated embodiment, the processor-based controller 121 is dedicated to performing the functional aspects of the technique disclosed herein and accessing display 130. However, in alternative embodiments, the processor-based control unit 120 may also more generally perform all control functions for the ECG monitor 109 in addition to the technique, system, and process disclosed herein. For example, in some embodiments, the processor-based control unit 120 may present a user interface (not separately shown) to a user on the display 130 by executing a set of user interface (“UI”) instructions 136 residing in memory 124 as shown in FIG. 1.

As those in the art having the benefit of this disclosure will appreciate, the term “processor” is understood in the art to have a definite connotation of structure. A processor may be hardware, software, or some combination of the two. In the illustrated embodiment of FIG. 2, the processor 200 is a programmed hardware processor, such as a controller, a microcontroller (“MP”) or a Central Processing Unit (“CPU”). However, in alternative embodiments, the processor 200 may be a Digital Signal Processor (“DSP”) [for example as provided by the Texas Instruments corporation], a processor chip set, an Application Specific Integrated Circuit (“ASIC”), an appropriately programmed Electrically Programmable Read-Only Memory (“EPROM”), an appropriately programmed Electrically Erasable, Programmable Read-Only Memory (“EEPROM”), a logic circuit, etc.

The processor 200 executes machine executable instructions 205 residing in the memory 210 to perform the software-implemented functionality of the techniques described herein. The instructions 205 may be embedded as firmware in the memory 210 or encoded as routines, subroutines, applications, etc. The memory 210, as well as the memory 124 in embodiments where they differ, may include Read-Only Memory (“ROM”), Random Access Memory (“RAM”), or a combination of the two. They will typically be installed memory but may be removable. They may be primary storage, secondary, tertiary storage, or some combination thereof implemented using electromagnetic, optical, or solid-state technologies.

Accordingly, in the illustrated embodiment, the processor-based control unit 120 performs the software-implemented functionality of the presently disclosed techniques using the mode adaptive coefficient filter system disclosed. More particularly, the processor 200 executes the instructions 205, both shown in FIG. 2, to perform the programmed functionality in which the PPM patient testing for ECG functions and monitoring process includes the disclosed techniques, components, and related functionalities to provide for an improved ECG artifact resolution.

Also accordingly, as those in the art having the benefit of this disclosure will appreciate, the precise makeup of the PPM product and processor 200 and sensor interface 118 may vary, sufficiently so that compact products may be contacted to a patent by a direct skin contacting interface on (FIG. 1) for ongoing monitoring.

The mode adaptive coefficient filters of the present disclosure have been designed and implemented in physiological patent monitoring PPM products and with an infinite impulse response (“IIR”) structure for the three (N) filter modes (but not limited to three), the function helps mitigate potential and actual physiological artifact risks and resolve system issues found in quality testing. The method disclosed herein can be utilized for both IIR and FIR systems with similar filter structures and with compatible digital coefficients in the active filters so that the parameter sets for the IIR and FIR filter systems would be of approximate equivalent size and tuned for particular filters coefficients to maintain filter function.

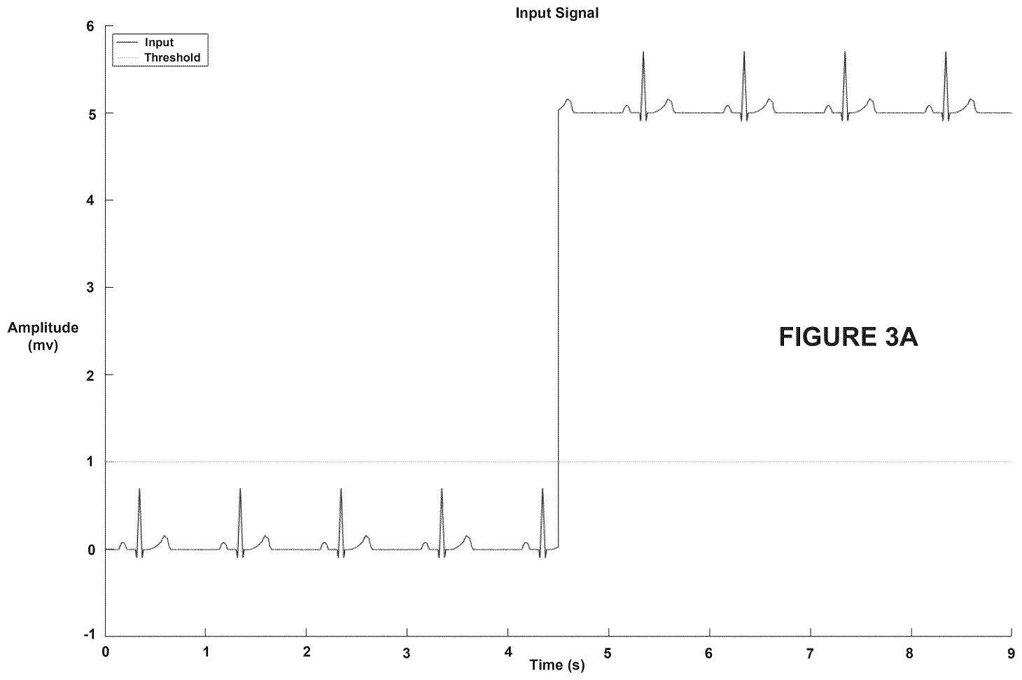

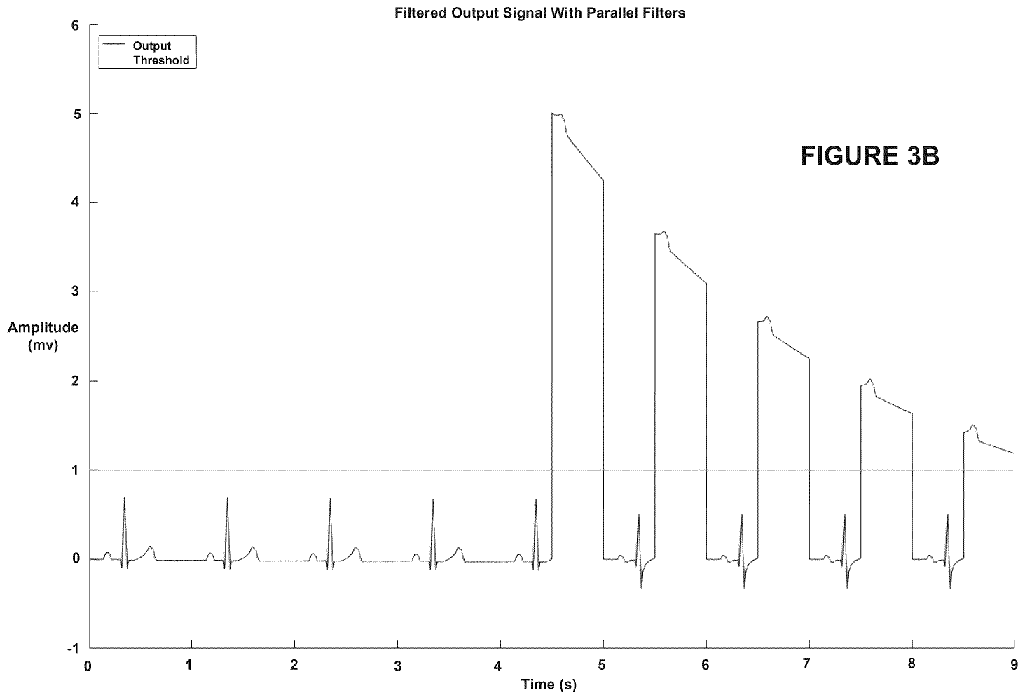

FIG. 3A-FIG. 3B conceptually illustrate a case where noticeable artifacts were observed on a system display without the mode adaptive coefficient filter design as disclosed herein, when a large ECG baseline offset was applied to the input signal and triggered a filter mode switching from a high-fidelity mode to a low fidelity mode in order to restore the baseline to a zero level. The signal that is evaluated (the “input signal”, or “ECG input signal”) is the output of the entire signal chain. This means that it is not practical to predict when to precisely switch back to the high-fidelity mode from the low-fidelity mode, as the combined response of the signal conditioning with the high-fidelity filter mode is unknown at that point. It is only after switching to high-fidelity mode that one can evaluate whether the combined response meets the baseline criteria.

To demonstrate the benefit of the MACF, FIG. 3A-FIG. 3B illustrates a conventional baseline artifact correction mechanism implementing a parallel filter structure. FIG. 3A shows an input signal and FIG. 3B shows an output signal in a prior art system using parallel filters. The input signal contains a baseline offset artifact, beginning at 4.5 s.

The system begins in high-fidelity mode. The system detects the baseline offset artifact at 5 s and switches to the low-fidelity parallel filter output to remove the baseline artifact. The combined response of the signal conditioning with the original filter mode cannot be determined until the system switches back to the original high-fidelity mode. At 5.5 s the system switches back to the high-fidelity mode to determine if the high-fidelity mode has removed the artifact. The artifact is still present at 5.5 s, and the system detects the baseline offset artifact again at 6 s and switches back to the low-fidelity mode to remove the baseline artifact on the display while the high-fidelity filter continues to settle. This cycle repeats past the end of the figure until the low-fidelity mode completes removing the artifact.

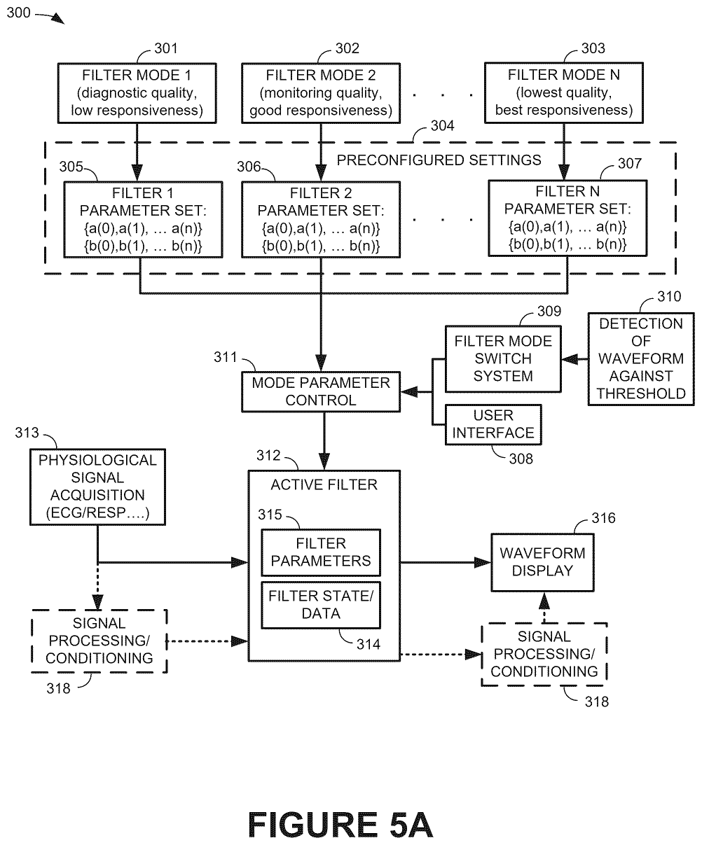

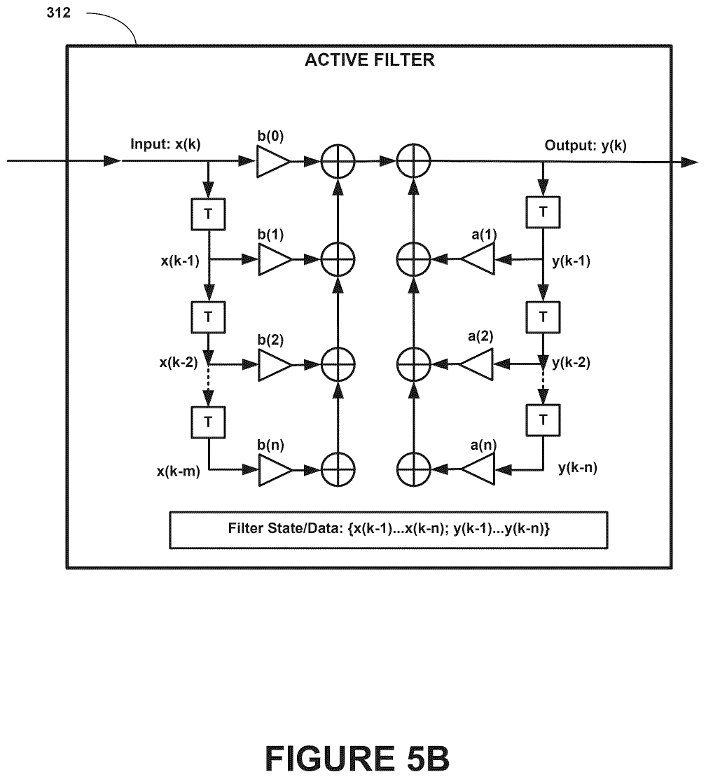

That, in turn, results in the oscillatory behavior as the signal chain repeatedly attempts to revert unsuccessfully to high-fidelity mode. One possible solution could be running multiple complete signal chains, but that is not possible due to computational resource limitations. In addition, the switch between the two complete chains could still result in a signal artifact due to the difference in filter characteristics between them. By sharing the history between the modes (FIG. 5A, 314), the high-fidelity mode picks up where the low-fidelity mode left off, allowing one to predict the initial combined response of the signal chain.

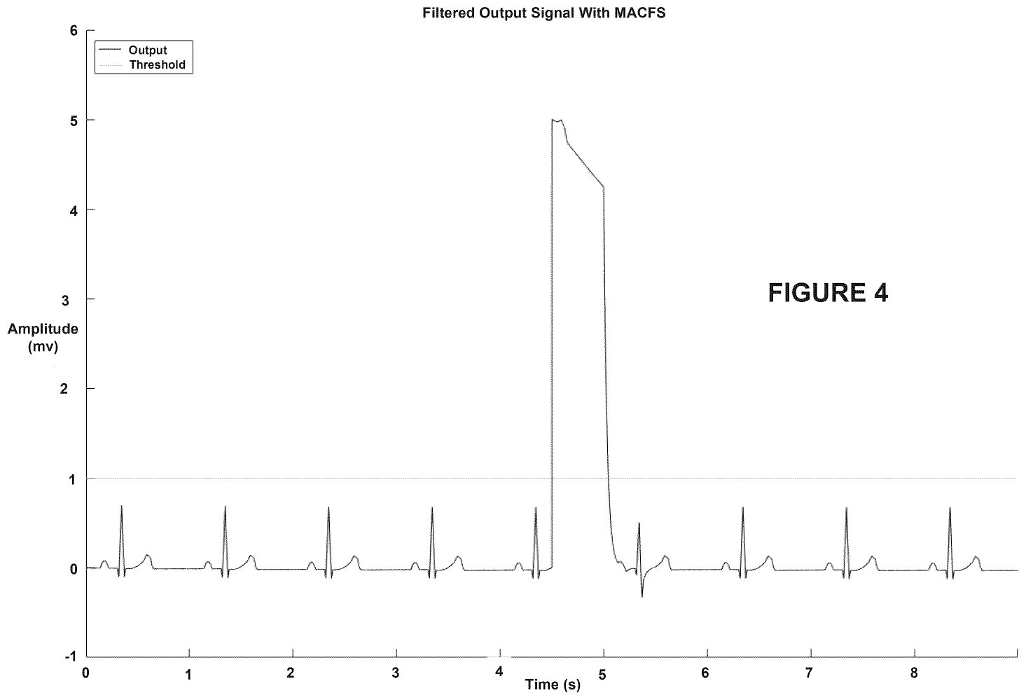

FIG. 4 conceptually illustrates application of the disclosed technique to solve this issue. The system begins in high-fidelity mode. It detects the baseline offset artifact at 5 s and engages the low-fidelity mode to remove the baseline offset artifact. Once the baseline offset artifact is removed, it switches back to the previous high-fidelity mode at 5.5 s. The high-fidelity mode slowly removes the artifact from 4.5 s to 5 s prior to the detection logic engaging the much faster low-fidelity mode at 5 s. Thus, as illustrated, when a large baseline offset occurs, no oscillation artifact was observed when switching filter modes, e.g., the ECG waveform output is returned without offsets for the same input ECG waveform. The presently claimed subject matter therefore removes at least one baseline offset artifact. The presently claimed subject matter removes at least one baseline offset artifact at a faster speed than prior known methods. The presently claimed subject matter provides an improvement over previous methods by preventing an oscillation artifact from the ECG waveform output.

FIGS. 5A and 5B conceptually illustrate the mode adaptive coefficient filter system 300 in block diagram form for a PPM patient monitoring product using an n-order IIR (infinite impulse response) filter as noted functions or modes. The mode adaptive coefficient filter system 300 may be implemented in software, hardware, or a combination thereof. In the illustrated embodiment, the implementation is in a combination of hardware and software. For example, in the mode adaptive coefficient filter system 300 of FIG. 5A, the active filter 312 may be implemented in hardware, e.g., an integrated circuit device of some kind. The setting of the filter parameters 315 may be software-implemented.

In general, the active filter 312 of the illustrated embodiment are implemented in digital filters, typically a hardware implementation, under the software control of the processor-based control unit 120. The hardware may be located in the processor-based control unit 120 or elsewhere in the ECG monitor 109. The software implemented aspects of the mode adaptive coefficient filter system 300 are, in the disclosed embodiments, implemented in the instructions 205 residing in the memory 120 of the processor-based control unit 120, all shown in FIG. 2. As will be described, the processor-based control unit 120 applies the mode adaptive coefficient filter system 300 through execution of the instructions 205.

Other embodiments may, however, differ. The implementation of the mode adaptive coefficient filter system 300 in terms of the allocation between software and hardware will be an implementation specific detail. The allocation may be made in considerations of factors such as the deign of the various aspects of the mode adaptive coefficient filter system 300, the available computing resources, physical constraints imposed by other aspects of the ECG monitor 109, etc. Those in the art having the benefit of this disclosure will be able to make such an allocation in light of those and other such considerations.

The mode adaptive coefficient filters are provided based on the clinical needs of a clinician to achieve a variety of physiological signal qualities for treatment, e.g., a diagnostic Filter Mode 1 as 301 (for diagnostic quality and low responsiveness), a monitoring Filter Mode 2 as 302 (for monitoring quality and good responsiveness) to other modes (shown as “ . . . ”) as a Filter Mode N as 303 (for lowest quality and reduced fidelity for best responsiveness and quick baseline restoring). The clinician designates the desired Filter Mode 301, 302, 303, based upon the clinical needs at that time based on processing response change rate and intended display response (faster, slower etc.). The clinical concerns are signal fidelity and quality without artifacts vs responsiveness of the display. It will be understood from FIG. 5A that multiple Figure Modes may be selected. Physiological signal quality for diagnostic monitoring is particularly of concern where there may be a very high overload of ECG signals during treatment and thus a very high baseline that needs to be urgently filtered to a diagnostically suitable baseline for the clinician to appreciate during real time treatment.

The preconfigured settings 304 provide respective filter parameters or digital coefficients in the respective Filter Mode settings for system 300, and that are preconfigured in system memory or system registers to achieve the intended respective Filter Mode and noted, respectively, as a Filter 1 Parameter Set 305, a Filter 2 Parameter Set 306 to a Filter N Parameter Set 307. The parameter sets contain the numerical values for each filter mode coefficient in the preconfigured settings 304 so as to maintain a consistent active filter state.

The filter mode can be selected (changed or switched) by either the user of system 300 via a User Interface 308 or by a filter mode switching system 309 which is an automatic and dynamic filter mode switching system selected when the signal waveform is displayed either in or out of designated thresholds (e.g., a defined area on the screen display) in Detection Threshold block 310.

Upon determining the selected mode filter setting parameters, a Mode Parameter Control function 311 with a plurality of coefficients and the preconfigured settings 304 and influenced by either User Interface 308 or Detection Threshold block 310 detection, will apply the selected mode filter parameters 315 to an Active Filter 312 which is a digital filter in which the internal coefficients are changed such that Active Filter 312 processes an input signal generated from a Physiological Signal Acquisition function 313 by using the filter state/data 314 in Active Filter 312 and outputting a Waveform Display 316 without artifacts or effort in signal display quality.

There is the single Active Filter 312 wherein system 300 changes the coefficient filters and modes in the Active Filter 312 with the primary benefit being minimized artifacts and oscillations in the signal results following those changes to the stored coefficient filters so there is per the Mode Parameter Control function 311 one state and one data set for the mode adaptive coefficient filter system that reduces or eliminates signal artifacts for either IIR or FIR filters systems with similar filter structures.

The present method and mode adaptive coefficient filter system provides that switching mode filter parameters while retaining the current filter history data will ensure a smooth and artifact-free transition in a resulting Waveform Display 316, for example, showing a continuous baseline despite changes in the active filter coefficients. The present method and mode adaptive coefficient filter system prevents instability and additional latency (such as oscillations or ripples) in real-time signal rendering to Waveform Display 316.

Furthermore, some embodiments may also employ an optional Signal Processing/Conditioning 318. This optional Signal Processing/Conditioning 318 may be omitted; or may be performed between Physiological Signal Acquisition function 313 and the active filter 312; or may be performed between the active filter 312 and the Waveform Display 316; or may be performed both between the Physiological Signal Acquisition function 313 and the active filter 312 and between the active filter 312 and the Waveform Display 316, depending on the individual embodiment. The addition of 318 complicates the prediction of the combined filter output 316. The disclosed technique employs a common filter state/data 314 to mitigate the challenge of predicting the combined filter output.

More particularly, there are two primary design constraints for these designs. First, additional signal processing occurs after the active filter. This filtering may be nonlinear, which makes it impractical to predict the contribution of inactive filter(s) to the response of the signal chain. Second, the settling time should be low. Without the first design constraint, it would be possible to evaluate the parallel case and avoid the oscillatory behavior. It would then not meet the second design constraint.

Additionally, as those in the art having the benefit of the disclosure will appreciate, Waveform Display 316 is an operative result to visual display 130 for a clinician when addressing a PPM patient monitoring product. Similarly, those in the art having further the benefit of this disclosure will recognize that the output of the resultant adaptive coefficient filter system need not be visual but may be digital to another destination designation such as a remote data node consumer (not shown) for optionally further algorithm processing free of the undesirable artifacts observed on a system without the present adaptive coefficient filter or to another remote data node for display.

Accordingly, as those in the art having the benefit of this disclosure will appreciate, the method and system can be utilized for both infinite impulse response IIR filters and finite impulse response FIR filters with similar filter structures.

Also accordingly, those in the art having the benefit of this disclosure will appreciate that use of PPM monitoring for ECG functions with an alarm generator will benefit with an improved stability and reduced latency in real time signal rendering and a resulting improvement in alarm functions.

A further secondary performance benefit of the provided mode adaptive coefficient filter system with an active filter, is appreciated in high sample data situations where there is a benefit in avoiding the need to maintain a data state for multiple parallel filters as well as not having to perform computations on each of a series of multiple parallel filters. There are both memory and CPU benefits to the present invention are in addition to the primary goal of reducing signal artifacts. Such processor and memory benefits for the present single active filter increases rapidly as the volume of signal date processed increases.

The foregoing outlines the features of several embodiments so that those of ordinary skill in the art may better understand various aspects of the present disclosure. Those of ordinary skill in the art should appreciate that they may readily use the present disclosure as a basis for designing or modifying other processes and structures for carrying out the same purposes and/or achieving the same advantages of various embodiments introduced herein. Those of ordinary skill in the art should also realize that such equivalent constructions do not depart from the spirit and scope of the present disclosure, and that they may make various changes, substitutions, and alterations herein without departing from the spirit and scope of the present disclosure.

Although the subject matter has been described in language specific to structural features or methodological acts, it is to be understood that the subject matter of the appended claims is not necessarily limited to the specific features or acts described above. Rather, the specific features and acts described above are disclosed as example forms of implementing at least some of the claims.

Various operations of embodiments are provided herein. The order in which some or all of the operations are described should not be construed to imply that these operations are necessarily order dependent. Alternative ordering will be appreciated having the benefit of this description. Further, it will be understood that not all operations are necessarily present in each embodiment provided herein. Also, it will be understood that not all operations are necessary in some embodiments.

It will be appreciated that layers, features, elements, etc., depicted herein are illustrated with particular dimensions relative to one another, such as structural dimensions or orientations, for example, for purposes of simplicity and ease of understanding and that actual dimensions of the same differ substantially from that illustrated herein, in some embodiments. Moreover, “exemplary” is used herein to mean serving as an example, instance, illustration, etc., and not necessarily as advantageous. As used in this application, “or” is intended to mean an inclusive “or” rather than an exclusive “or”. In addition, “a” and “an” as used in this application and the appended claims are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, or variants thereof are used, such terms are intended to be inclusive in a manner similar to the term “comprising”. Also, unless specified otherwise, “first,” “second,” or the like are not intended to imply a temporal aspect, a spatial aspect, an ordering, etc. Rather, such terms are merely used as identifiers, names, etc. for features, elements, items, etc. For example, a first element and a second element generally correspond to element A and element B or two different or two identical elements or the same element.

This concludes the detailed description. The particular examples disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular examples disclosed above may be altered or modified and all such variations are considered within the scope and spirit of the invention. Accordingly, the protection sought herein is as set forth in the claims below.

1. An electrocardiogram (“ECG”) monitor, comprising:
a mode adaptive coefficient filter system for real time electrocardiogram (“ECG”) waveform processing of ECG waveforms received from a plurality of ECG electrodes, the mode adaptive coefficient filter system including at least one active n-order impulse filter, the n-order impulse filter being one of an infinite impulse response (“IIR”) filter and a finite impulse response (“FIR”) filter;
a display;
a processor-based control unit programmed to:
monitor a plurality of ECG electrodes when the ECG electrodes are ostensibly electrically connected;
receive a physiological signal acquired from one of the ECG electrodes as an electrical input signal including at least one artifact relative to a waveform display baseline;
apply the mode adaptive coefficient filter system based on a plurality of preconfigured filter parameter settings; and
render an input signal waveform in the display that removes the at least one artifact relative to the waveform display baseline.

2. The ECG monitor of claim 1, further comprising the plurality of ECG electrodes.

3. The ECG monitor of claim 1, further comprising:
a processor; and
a memory in which resides a plurality of instructions with which the processor-based control unit is programmed and that, when executed by the processor, cause the processor to apply the mode adaptive coefficient filter system based on a plurality of preconfigured filter parameter settings.

4. The ECG monitor of claim 1, wherein the processor-based control unit is further programmed to apply the mode adaptive coefficient filter based on a designated filter mode parameter control if the input signal exceeds a predetermined threshold.

5. The ECG monitor of claim 4, wherein:
the filter mode parameter control includes a selection from a filter mode switch system or a user interface; and
the filter mode switch system determines the mode parameter control and the mode adaptive coefficient filter when the input signal waveform is displayed out of the predetermined threshold.

6. The ECG monitor of claim 5, wherein the processor-based control unit is further programmed to:
determine the rendered input signal waveform display exceeds the predetermined threshold; and
issue an alarm.

7. The ECG monitor of claim 6, wherein the alarm is an audio alarm, a visual alarm on the display, or a combination of the audio alarm and the visual alarm.

8. A method for real time electrocardiogram (“ECG”) waveform processing, comprising:
monitoring a plurality of electrocardiogram (“ECG”) electrodes in a physiological patent monitoring (“PPM”) product ostensibly electrically connected to a human body and including an active n-order impulse filter, the n-order impulse filter being one of an infinite impulse response (“IIR”) filter and a finite impulse response (“FIR”) filter;
receiving a physiological signal acquired from one of the ECG electrodes as an electrical input signal including at least one artifact relative to a waveform display baseline;
applying a predetermined mode adaptive coefficient filter based on a plurality of preconfigured filter parameter settings;
rendering an input signal waveform in a display; and
removing the at least one artifact relative to the waveform display baseline.

9. The method of claim 8, wherein applying the predetermined mode adaptive coefficient filter includes applying the predetermined mode adaptive coefficient filter based on a designated filter mode parameter control if the input signal exceeds a predetermined threshold.

10. The method of claim 9, wherein:
ascertaining the filter mode parameter control includes a selection from a filter mode switch system or a user interface; and
the filter mode switch system determines the mode parameter control and the mode adaptive coefficient filter when the input signal waveform is displayed out of the predetermined threshold.

11. The method of claim 10 further comprising determining if the rendered input signal waveform display exceeds the predetermined threshold, issuing an alarm.

12. The method of claim 11, wherein issuing the alarm includes sounding an audio alarm, presenting a visual alarm on the display; or a combination of sounding the audio alarm and presenting the visual alarm on the display.

13. A non-transitory computer readable medium encoded with instructions that, when executed by a processor, perform a method for real time electrocardiogram (“ECG”) waveform processing, comprising:
monitoring a plurality of electrocardiogram (“ECG”) electrodes in a physiological patent monitoring (“PPM”) product ostensibly electrically connected to a human body and including an active n-order impulse filter;
the n-order impulse filter being one of an infinite impulse response (“IIR”) filter and a finite impulse response (“FIR”) filter;
receiving a physiological signal acquired from one of the ECG electrodes as an electrical input signal including at least one artifact relative to a waveform display baseline;
applying a predetermined mode adaptive coefficient filter based on a plurality of preconfigured filter parameter settings;
rendering an input signal waveform in a display; and
removing the at least one artifact relative to the waveform display baseline.

14. The non-transitory computer readable medium, of claim 13, wherein applying a predetermined mode adaptive coefficient filter includes applying the predetermined mode adaptive coefficient filter based on a designated filter mode parameter control if the input signal exceeds a predetermined threshold.

15. The non-transitory computer readable medium, of claim 14, wherein:
ascertaining the filter mode parameter control includes a selection from a filter mode switch system or a user interface; and
the filter mode switch system determines the mode parameter control and the mode adaptive coefficient filter when the input signal waveform is displayed out of the predetermined threshold.

16. The non-transitory computer readable medium, of claim 15, wherein the instructions further include, when executed by the processor, determining if the rendered input signal waveform display exceeds the predetermined threshold and issuing an alarm.

17. The non-transitory computer readable medium, of claim 16, wherein the instructions further include, when executed by the processor, issuing the alarm by sounding an audio alarm; or presenting a visual alarm on the display; or a combination of sounding the audio alarm and presenting the visual alarm on the display.

18. A mode adaptive coefficient filter system for real time electrocardiogram (“ECG”) waveform processing, comprising:
a plurality of electrocardiogram (“ECG”) electrodes in a physiological patient monitoring (“PPM”) product ostensibly electrically connected to a human body;
an ECG monitor electrically connected to the ECG electrodes, the ECG monitor including:
at least one active n-order impulse filter, the n-order impulse filter being one of an infinite impulse response (“IIR”) filter and a finite impulse response (“FIR”) filter;
a display; and
a processor-based control unit including:
a processor; and
a memory in which resides a plurality of instructions with which the processor-based control unit is programmed and that, when executed by the processor, cause the processor to apply the mode adaptive coefficient filter system based on a plurality of preconfigured filter parameter settings.

19. The system of claim 18, wherein the processor-based control unit is programmed to:
monitor the ECG electrodes when the ECG electrodes are ostensibly electrically connected;
receive a physiological signal acquired from one of the ECG electrodes as an electrical input signal including at least one artifact relative to a waveform display baseline;
apply the mode adaptive coefficient filter based on a plurality of preconfigured filter parameter settings; and
render an input signal waveform in the display that removes the at least one artifact relative to the waveform display baseline.

20. The system of claim 19, wherein the processor-based control unit further programmed to apply the predetermined mode adaptive coefficient filter based on a designated filter mode parameter control if the input signal exceeds a predetermined threshold.

21. The system of claim 20, wherein:
the filter mode parameter control includes a selection from a filter mode switch system or a user interface; and
the filter mode switch system determines the mode parameter control and the mode adaptive coefficient filter when the input signal waveform is displayed out of the predetermined threshold.
